(19) United States
(12) Patent Application Publication
(10) Pub. No.: US 2024/0296962 A1
(43) Pub. Date: Sep. 5, 2024

(54) NEUROTRANSMITTER CONCENTRATION MEASURING APPARATUS FOR SIMULTANEOUSLY PROVIDING LONG TIME MEASURING RESULTS OF CONCENTRATION FOR VARIOUS NEUROTRANSMITTER BASED ON FAST-SCAN CYCLIC VOLTAMMETRY AND METHOD THEREOF

(71) Applicant: Daegu Gyeongbuk Institute of Science and Technology, Daegu (KR)

(72) Inventors: Ji Woong CHOI, Seoul (KR); Seong Tak Kang, Yeosu-si (KR); Yun Ho Jeong, Seoul (KR); Eun Ho Kim, Daegu (KR)

(73) Assignee: Daegu Gyeongbuk Institute of Science and Technology, Daegu (KR)

(21) Appl. No.: 18/594,719

(22) Filed: Mar. 4, 2024

(30) Foreign Application Priority Data
Mar. 3, 2023 (KR) 10-2023-0028397

(51) Int. Cl. G16H 50/70

(52) U.S. Cl. CPC G16H 50/70

(57) ABSTRACT

A neurotransmitter concentration measuring apparatus includes a data collecting unit configured to collect fast-scan cyclic voltammetry (FSCV) data where capacitive charging current is included in faradaic current varying depending on injection concentration for each of multiple neurotransmitters, a data processing unit configured to process the FSCV data as second-derivative-based background removal (SDBR) data in a faradaic current form where the charging current is excluded, based on a second derivative for voltage of an individual voltammogram generated for each scan by background subtraction in the FSCV data, a deep learning processing unit configured to build a deep learning model that simultaneously estimates the concentration of the multiple neurotransmitters by learning the SDBR data with a deep learning network and a measurement result providing unit configured to simultaneously provide concentration measurement results of a neurotransmitter varying depending on real-time injection for each of the multiple neurotransmitters based on the learning model.

### TECHNICAL FIELD

The present invention relates to a deep learning-based neurotransmitter concentration measuring apparatus and method for simultaneously providing long-time concentration measurement results of various neurotransmitters based on fast-scan cyclic voltammetry (FSCV), and more particularly, to technology for building a deep learning model by learning second-derivative-based background removal (SDBR) data that contains concentration information regardless of time, with a deep learning network according to the concentration for each of various neurotransmitters, estimating in real time concentration measurement results of various neurotransmitters based on the built deep learning model, and simultaneously providing the estimated concentration measurement results.

### RELATED ART

Neurotransmitters play an important role in regulating various brain functions in the brain.

Improper signaling of neurotransmitters causes serious diseases, such as depression and movement disorder.

Dopamine that acts as a neurotransmitter in the brain is a neuroregulatory that transmits important information, such as cognition, reward and pleasure, and voluntary exercise, and dysregulation of a dopamine system is associated with the wide range of brain disorders, such as Parkinson's disease, Tourette syndrome, addiction, and schizophrenia.

Dopamine levels in target areas of the brain show highly dynamic changes and fluctuate over a variety of time scales.

Quantitative analysis of dopamine levels is very important in understanding the functional role of dopamine dynamics in the normal brain and studying brain disorder pathology in preclinical and clinical research.

To analyze and appropriately manage neurotransmitter dynamics, it is essential to develop technology for monitoring phasic and tonic levels of various neurotransmitters.

A phasic level indicates a change within seconds and a tonic level indicates a change within minutes to hours.

Microdialysis was used to measure tonic levels of multiple neurotransmitters in the brain.

Microdialysis may monitor multiple neurotransmitters in the brain, but has low temporal resolution (one sample per few minutes) to analyze the rapid dynamics of neurotransmitters.

This limitation of microdialysis prompted the development of fast-scan cyclic voltammetry (FSCV) that is high temporal resolution (10 Hz) technology.

FSCV was used to monitor phasic levels of neurotransmitters in the brain with high sensitivity.

However, since FSCV measurement continuously generates background charging current, FSCV data may be hard to analyze for a long period; 1 minute or more.

This continuous rise in current due to the background charging current is called background drift and standard FSCV technology had difficulty in measuring a tonic neurotransmitter level for more than 1 minute.

Although several studies have attempted to measure tonic levels using modified FSCV, most studies estimated tonic dopamine levels, abandoning high temporal resolution that is an advantage of FSCV.

Second-derivative-based background drift removal (SDBR) refers to second-derivative-based background drift removal technology and extracts tonic concentration information of neurotransmitters from each voltammogram with 10 Hz sampling frequency. SDBR can simultaneously measure a change in tonic dopamine and a change in phasic dopamine, but can not simultaneously measure neurotransmitters other than dopamine.

That is, the most recent technology, SDBR, measures a dopamine level for a long time, but still measures only concentration of a single neurotransmitter, which may be a limitation of the related art that can not simultaneously observe two or more neurotransmitters with a single electrode.

The recent study showed that a deep learning approach is superior to a linear model when estimating the phasic concentration of four neurotransmitters using a voltammogram of FSCV. However, it is confirmed that it is impossible to estimate tonic levels of neurotransmitters since learned FSCV data includes only phase signal information.

### DETAILED DESCRIPTION

### Subject

The present invention is to build a deep learning model by training second-derivative-based background removal (SDBR) data that contains concentration information regardless of time, with a deep learning network according to the concentration for each of various neurotransmitters, to estimate in real time concentration measurement results of various neurotransmitters based on the built deep learning model, and to simultaneously provide the estimated concentration measurement results.

The present invention is to continuously increase the number of simultaneously measurable neurotransmitters through pre-training by enabling simultaneous estimation after deep learning model training for all neurotransmitters that can be measured in FSCV data. The present invention is to provide an ideal measurement environment when trying to understand a specific brain disease or brain function and to simultaneously observe roles of various neurotransmitters.

The present invention is to provide a deep learning-based neurotransmitter concentration measuring apparatus and method for simultaneously providing long-time concentration measurement results of various neurotransmitters based on FSCV data that can measure various neurotransmitters over a long time with high temporal resolution (0.1 seconds).

The present invention is to improve a detailed analysis rate of signals of neurotransmitters since temporal resolution is improved as a form of faradaic current of a neurotransmitter is extractable regardless of capacitive charging current.

### Solution

A neurotransmitter concentration measuring apparatus according to an example embodiment of the present invention may include a data collecting unit configured to collect fast-scan cyclic voltammetry (FSCV) data in which capacitive charging current is included in faradaic current varying depending on injection concentration for each of multiple neurotransmitters; a data processing unit configured to process the FSCV data as second-derivative-based background removal (SDBR) data in a faradaic current form in which the capacitive charging current is excluded, based on a second derivative for voltage of an individual voltammogram generated for each scan by background subtraction in the FSCV data; a deep learning processing unit configured to build a deep learning model that simultaneously estimates the concentration of the multiple neurotransmitters by learning from the SDBR data with a deep learning network; and a measurement result providing unit configured to simultaneously provide concentration measurement results of varying neurotransmitters based on the deep learning model.

The deep learning processing unit may be configured to build the deep learning model that simultaneously estimates the concentration of the multiple neurotransmitters when SDBR data based on FSCV data measured for the multiple neurotransmitters is applied to the deep learning model trained by SDBR data that is measured and processed in advance for the multiple neurotransmitters with the deep learning network.

The multiple neurotransmitters may include at least two of dopamine, epinephrine, norepinephrine, and serotonin.

The data processing unit is configured to process the FSCV data as the SDBR data by extracting, from the FSCV data, the individual voltammogram in which the faradaic current and the capacitive charging current are measured together for each scan by the background subtraction in relation to a peak according to neurotransmitter injection, by multiplying voltage of the extracted individual voltammogram by a negative value after the second derivative, and by quantifying a curvature of a neurotransmitter peak.

The extracted individual voltammogram includes phasic measurement results in relation to the neurotransmitter concentration measurement, and the SDBR data may include the phasic measurement results and tonic measurement results in relation to the neurotransmitter concentration measurement.

The data processing unit may be configured to extract the individual voltammogram such that a voltammogram around a neurotransmitter oxidation peak after the background subtraction has a symmetrical Gaussian shape.

The data processing unit may be configured to process the SDBR data such that amplitude current of a neurotransmitter oxidation peak of a voltammogram corresponding to the individual voltammogram has a linear correlation with the concentration of a neurotransmitter by eliminate background charging current generated around the neurotransmitter oxidation peak irrelevant to neurotransmitters.

The measurement result providing unit may be configured to determine a neurotransmitter oxidation peak voltage based on the faradaic current form in which the capacitive charging current based on the SDBR data is removed and to provide the concentration of a neurotransmitter compared to the determined neurotransmitter oxidation peak voltage as the concentration measurement results of the neurotransmitter.

The data collecting unit may be configured to collect the FSCV data in which the faradaic current increasing at an injection point in time of the neurotransmitter and the capacitive charging current gradually increasing over time are combined.

A neurotransmitter concentration measuring method according to an example embodiment of the present invention may include collecting, by a data collecting unit, fast-scan cyclic voltammetry (FSCV) data in which capacitive charging current is included in faradaic current varying depending on injection concentration for each of multiple neurotransmitters; processing, by a data processing unit, the FSCV data as second-derivative-based background removal (SDBR) data in a faradaic current form in which the capacitive charging current is removed, based on a second derivative for voltage of an individual voltammogram generated for each scan by background subtraction in the FSCV data; building, by a deep learning processing unit, a deep learning model that simultaneously estimates the concentration of the multiple neurotransmitters by training the SDBR data with a deep learning network; and simultaneously providing, by a measurement result providing unit, concentration measurement results of a neurotransmitter varying depending on real-time injection for each of the multiple neurotransmitters based on the deep learning model.

The building of the deep learning model that simultaneously estimates the concentration of the multiple neurotransmitters by training the SDBR data with the deep learning network may include building the deep learning model that simultaneously estimates the concentration of the multiple neurotransmitters when SDBR data based on FSCV data additionally measured for one neurotransmitter among the multiple neurotransmitters is applied to the deep learning model built by training SDBR data that is measured and processed in advance for the multiple neurotransmitters with the deep learning network.

The multiple neurotransmitters may include at least two of dopamine, epinephrine, norepinephrine, and serotonin.

The processing of the FSCV data as the SDBR data in the faradaic current form in which the capacitive charging current is removed, based on the second derivative for voltage of the individual voltammogram generated for each scan by background subtraction in the FSCV data may include processing the FSCV data as the SDBR data by extracting, from the FSCV data, the individual voltammogram in which the faradaic current and the capacitive charging current are reflected together for each scan by the background subtraction in relation to a peak according to neurotransmitter injection, by multiplying voltage of the extracted individual voltammogram by a negative value after the second derivative, and by quantifying a curvature of a neurotransmitter peak.

The extracted individual voltammogram may include phasic measurement results in relation to the neurotransmitter concentration measurement, and the SDBR data may include the phasic measurement results and tonic measurement results in relation to the neurotransmitter concentration measurement.

### Effect

The present invention can build a deep learning model by learning second-derivative-based background removal (SDBR) data that contains concentration information regardless of time, with a deep learning network according to the concentration for each of various neurotransmitters, can estimate in real time concentration measurement results of various neurotransmitters based on the built deep learning model, and can simultaneously provide the estimated concentration measurement results.

The present invention can continuously increase the number of simultaneously measurable neurotransmitters through pre-training by enabling simultaneous estimation after deep learning model training for all neurotransmitters that can be measured in FSCV data.

The present invention can provide an ideal measurement environment when trying to understand a specific brain disease or brain function and to simultaneously observe roles of various neurotransmitters.

The present invention can provide a deep learning-based neurotransmitter concentration measuring apparatus and method for simultaneously providing long-time concentration measurement results of various neurotransmitters based on FSCV data that may measure various neurotransmitters over a long time with high temporal resolution (0.1 seconds).

The present invention can improve a detailed analysis rate of a signal of dopamine that is a neurotransmitter since temporal resolution is improved as a form of faradaic current of a neurotransmitter is extractable regardless of capacitive charging current.

### BRIEF DESCRIPTION OF DRAWINGS

FIG. 1 is a diagram describing a process of processing, by a neurotransmitter concentration measuring apparatus, second-derivative-based background drift removal (SDBR) data in relation to neurotransmitter concentration according to an example embodiment of the present invention.

FIG. 2 is a diagram describing a neurotransmitter concentration measuring apparatus according to an example embodiment of the present invention.

FIG. 3 is a diagram describing a procedure of a neurotransmitter concentration measuring method that builds a deep learning model by deep-learning SDBR data and simultaneously measures the concentration of various neurotransmitters based on the deep learning model according to an example embodiment of the present invention.

FIG. 4 is a diagram describing a concentration measurement experiment of neurotransmitters based on a deep learning model according to an example embodiment of the present invention.

FIGS. 5A to 5D are graphs describing concentration measurement experiment results of neurotransmitters based on a deep learning model according to an example embodiment of the present invention.

FIG. 6 is a diagram describing a deep learning model according to an example embodiment of the present invention.

FIGS. 7 and 8 are flowcharts illustrating a neurotransmitter concentration measuring method according to an example embodiment of the present invention.

### MODE

The following structural or functional descriptions of example embodiments according to the concept of the present invention described herein are merely intended for the purpose of describing the example embodiments according to the concept of the present invention and the example embodiments according to the concept of the present invention may be implemented in various forms and are not construed as limited to the example embodiments described herein.

Various modifications and various forms may be made to the example embodiments according to the concept of the present invention and thus, the example embodiments are illustrated in the drawings and the present specification is described in detail. However, it should be understood that the example embodiments according to the concept of the present invention are not construed as limited to specific implementations and should be understood to include all changes, equivalents, and replacements within the idea and the technical scope of the present invention.

Although terms of “first,” “second,” and the like are used to explain various components, the components are not limited to such terms. These terms are used only to distinguish one component from another component. For example, a first component may be referred to as a second component, or similarly, the second component may be referred to as the first component without departing from the scope according to the concept of the present invention.

When it is mentioned that one component is “connected” or “accessed” to another component, it may be understood that the one component is directly connected or accessed to another component or that another component is interposed between the two components. In addition, when it is described that one component is “directly connected” or “directly joined” to another component, it should be understood that still another component is absent therebetween. Likewise, expressions, for example, “between” and “immediately between” and “immediately adjacent to” may also be construed as described in the foregoing.

The terminology used herein is for the purpose of describing particular example embodiments only and is not to be limiting of the present invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, stages, operations, elements, components or combinations thereof, but do not preclude the presence or addition of one or more other features, integers, stages, operations, elements, components, or combinations thereof.

Unless otherwise defined herein, all terms used herein including technical or scientific terms have the same meanings as those generally understood by one of ordinary skill in the art. Terms defined in dictionaries generally used should be construed to have meanings matching contextual meanings in the related art and are not to be construed as an ideal or excessively formal meaning unless otherwise defined herein.

Hereinafter, example embodiments will be described in detail with reference to the accompanying drawings. However, the scope of the claims is not limited to or restricted by the example embodiments. Like reference numerals presented in the respective drawings refer to like components throughout.

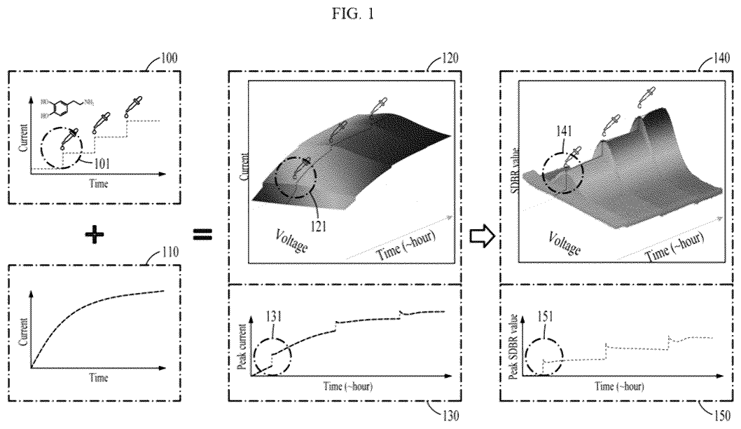

FIG. 1 is a diagram describing a process of processing, by a neurotransmitter concentration measuring apparatus, second-derivative-based background drift removal (SDBR) data in relation to neurotransmitter concentration according to an example embodiment of the present invention.

FIG. 1 illustrates a process of processing, by a neurotransmitter concentration measuring apparatus, second-derivative-based background drift removal (SDBR) data in relation to neurotransmitter concentration according to an example embodiment of the present invention.

That is, to extract faradaic current excluding capacitive charging current of which long-time measurement was impossible using the existing fast-scan cyclic voltammetry (FSCV) data, FIG. 1 illustrates a process of extracting SDBR data in a faradaic current form in which capacitive charging current is excluded through a second derivative after background subtraction in FSCV data.

Referring to FIG. 1, a graph 100 shows faradaic current according to injection of dopamine that is a neurotransmitter over time and a graph 110 shows capacitive charging current over time.

Also, a graph 120 is three-dimensional and shows an individual voltammogram that is the result of applying background subtraction for each scan to FSCV data in which current based on the graph 100 and current based on the graph 110 are combined, and a graph 130 illustrates the same two-dimensionally.

Meanwhile, a graph 140 is three-dimensional and shows SDBR data that quantifies and expresses a curvature of a neurotransmitter peak by multiplying data of the graph 120 by a negative value after the second derivative and a graph 150 illustrates the same two-dimensionally.

A point 101 in the graph 100 represents an injection point in time of dopamine that is a neurotransmitter, and corresponds to a point 121 in the graph 120, a point 131 in the graph 130, a point 141 in the graph 140, and a point 151 in the graph 150.

However, comparing the point 101 to the point 131 and the point 151, it can be seen that the point 101 and the point 151 are similar and there is a difference between the point 131 and the point 101 due to the effect of capacitive charging current.

This is because the neurotransmitter concentration measuring apparatus according to an example embodiment of the present invention processes the SDBR data as a result of applying an SDBR technique of multiplying data of the point 131 by a negative value after a second derivative and provides data on the graph 150 that includes the point 151 as a result of processing the SDBR data as the faradaic current in which the capacitive charging current is removed.

An SDBR data processing method can model a background-subtracted voltammogram around a neurotransmitter oxidation peak generated for each scan and can extract a specific scan time and a dopamine oxidation peak voltage from the modeled voltammogram through Equation 1 below.

$${Voltgram}_{BS} ( V , t ) = {e}^{- \frac{{( V - {peak}_{v} )}^{2}}{2}} {Conc}_{DA} + {Charg}_{c} ( t ) \qquad [ Equation 1 ]$$

In Equation 1, Voltgram_{BS} represents a voltammogram to which a background subtraction is applied, V represents a surrounding voltage, t represents a specific scan time, peakᵥ represents a dopamine oxidation peak, Conc_{DA} represents dopamine concentration, and Charge denotes background charging current.

Dopamine oxidation peak current of a voltammogram may be observed by setting V to peakᵥ and the corresponding equation may be expressed as Equation 2 below.

$${Voltgram}_{BS} ( {peak}_{v} , t ) = {Conc}_{DA} + {Charg}_{c} ( t ) \qquad [ Equation 2 ]$$

In Equation 2 that represents the voltammogram in which current of peakᵥ is removed from general background, Charge may be maintained constant.

If Charge is removed, the intrinsic curvature of the dopamine oxidation peak may be quantified by applying a second derivative to each background-subtracted voltammogram, which may be expressed as Equation 3 below.

$${Voltgram}_{SDBR} ( V , t ) = \frac{- {d}^{2}}{{dV}^{2}} {Voltgram}_{BS} ( V , t ) = ( 1 - {( V - {peak}_{v} )}^{2} ) {e}^{- \frac{{( V - {peak}_{v} )}^{2}}{2}} {Conc}_{DA} \qquad [ Equation 3 ]$$

In Equation 3, VoltgramSDBR refers to SDBR data and represents a voltammogram to which SDBR is applied.

The dopamine peak current may be observed after the second derivative of the voltammogram modeled by setting V to peakᵥ, which may be expressed as Equation 4 below.

$$\frac{- {d}^{2}}{{dV}^{2}} {Voltgram}_{BS} ( {peak}_{v} , t ) = {Conc}_{DA} \qquad [ Equation 4 ]$$

According to Equation 4, it can be seen that the capacitive charging current is removed from the individual voltammogram in which the background is subtracted in the FSCV data and only faradaic current remains.

According to an example embodiment of the present invention, to extract faradaic current excluding capacitive charging current of which long-time measurement was impossible using the existing FSCV data, the neurotransmitter concentration measuring apparatus can extract a form of neurotransmitter faradaic current regardless of the capacitive charging current by multiplying voltage in an individual voltammogram by a negative value after a second derivative and by quantifying a curvature of a neurotransmitter peak.

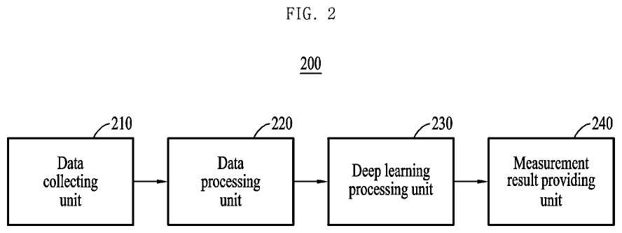

FIG. 2 is a diagram describing a neurotransmitter concentration measuring apparatus according to an example embodiment of the present invention.

FIG. 2 illustrates a component of a neurotransmitter concentration measuring apparatus according to an example embodiment of the present invention.

Referring to FIG. 2, a neurotransmitter concentration measuring apparatus 200 according to an example embodiment of the present invention includes a data collecting unit 210, a data processing unit 220, a deep learning processing unit 230, and a measurement result providing unit 240.

According to an example embodiment of the present invention, the data collecting unit 210 may collect fast-scan cyclic voltammetry (FSCV) data in which capacitive charging current is reflected in faradaic current varying depending on injection concentration for each of multiple neurotransmitters.

For example, the data collecting unit 210 may collect the FSCV data in which the faradaic current increasing at an injection point in time of the neurotransmitter and the capacitive charging current gradually increasing over time are combined.

For example, the multiple neurotransmitters may include at least two of dopamine, epinephrine, norepinephrine, and serotonin.

The multiple neurotransmitters are not limited to the aforementioned substances and include all neurotransmitters that allow measurement of FSCV data.

According to an example embodiment of the present invention, the data processing unit 220 can process the FSCV data as second-derivative-based background removal (SDBR) data in a faradaic current form in which the capacitive charging current is excluded, based on a second derivative for voltage of an individual voltammogram generated for each scan by background subtraction in the FSCV data.

For example, the data processing unit 220 can process the FSCV data as the SDBR data by extracting, from the FSCV data, the individual voltammogram in which the faradaic current and the capacitive charging current are reflected together for each scan by background subtraction in relation to a peak according to neurotransmitter injection, by multiplying voltage of the extracted individual voltammogram by a negative value after the second derivative, and by quantifying a curvature of a neurotransmitter peak.

For example, the extracted individual voltammogram may include phasic measurement results in relation to neurotransmitter concentration measurement.

Meanwhile, the SDBR data may include the phasic measurement results and tonic measurement results in relation to the neurotransmitter concentration measurement.

For example, the phasic measurement results may be results for a change in concentration that varies in seconds and the tonic measurement results may be results for a change in concentration that varies in minutes to hours. That is, the tonic measurement results may be concentration change results measured for a long period of time.

According to an example embodiment of the present invention, the data processing unit 220 can extract an individual voltammogram such that a voltammogram around a neurotransmitter oxidation peak after the background subtraction has a symmetrical Gaussian shape.

For example, the data processing unit 220 can process the SDBR data such that amplitude current of a neurotransmitter oxidation peak of a voltammogram corresponding to an individual voltammogram has a linear correlation with the concentration of a neurotransmitter and background charging current generated around the neurotransmitter oxidation peak is irrelevant to voltage.

According to an example embodiment of the present invention, the deep learning processing unit 230 can build a deep learning model that simultaneously estimates the concentration of the multiple neurotransmitters by learning the SDBR data with a deep learning network.

For example, the deep learning processing unit 230 can build a deep learning model that simultaneously estimates the concentration of multiple neurotransmitters when SDBR data based on FSCV data additionally measured for one neurotransmitter among the multiple neurotransmitters is applied to a deep learning model built by learning SDBR data that is measured and processed in advance for the multiple neurotransmitters with a deep learning network.

According to an example embodiment of the present invention, the deep learning model may simultaneously estimate changes in concentration of various neurotransmitters over a long time.

The deep learning model supports long-time measurement of at least two neurotransmitters with high temporal resolution to estimate the accurate concentration of various neurotransmitters over a long time.

According to an example embodiment of the present invention, the measurement result providing unit 240 can simultaneously provide concentration measurement results of a neurotransmitter varying depending on real-time injection with respect to each of multiple neurotransmitters based on the deep learning model.

For example, the measurement result providing unit 240 can determine a neurotransmitter oxidation peak voltage based on a faradaic current form in which capacitive charging current based on SDBR data is excluded and can provide the concentration of a neurotransmitter compared to the determined neurotransmitter oxidation peak voltage as concentration measurement results of the neurotransmitter.

According to an example embodiment of the present invention, the measurement result providing unit 240 can provide concentration measurement results by estimating SDBR data that contains concentration information regardless of time in a not-trained electrode and environment based on a deep learning model trained according to concentration for each substance.

Therefore, the present invention can build a deep learning model by learning SDBR data that contains concentration information regardless of time with a deep learning network according to concentration for each of various neurotransmitters, can estimate in real time concentration measurement results of various neurotransmitters based on the built deep learning model, and can simultaneously provide the estimated concentration measurement results.

Also, the present invention can provide an ideal measurement environment when trying to understand a specific brain disease or brain function and to simultaneously observe roles of various neurotransmitters.

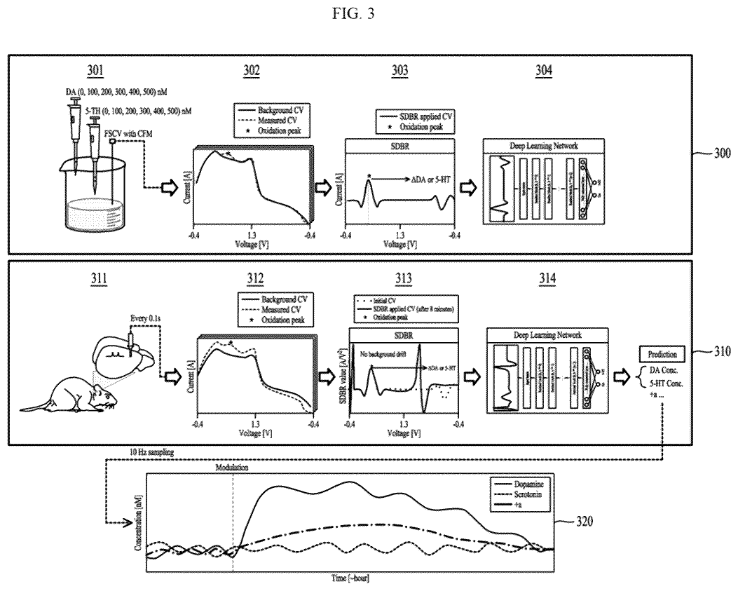

FIG. 3 is a diagram describing a procedure of building, by a neurotransmitter concentration measuring apparatus, a deep learning model by deep learning SDBR data and simultaneously measuring the concentration of various neurotransmitters based on the deep learning model according to an example embodiment of the present invention.

FIG. 3 illustrates a procedure of a neurotransmitter concentration measuring method that builds a deep learning model by deep learning SDBR data and simultaneously measures the concentration of various neurotransmitters based on the deep learning model according to an example embodiment of the present invention.

Referring to FIG. 3, a neurotransmitter concentration measuring method according to an example embodiment of the present invention performs a deep learning model building procedure 300 and a procedure 310 of simultaneously providing results of measuring the concentration of various neurotransmitters using a built deep learning model.

Initially, describing the deep learning model building procedure 300, the neurotransmitter concentration measuring method measures FSCV for variety of concentration of various neurotransmitters to generate learning data in operation 301.

In operation 302, the neurotransmitter concentration measuring method provides voltammograms before and after injecting a neurotransmitter related drug in relation to the FSCV measured in operation 301.

In operation 303, the neurotransmitter concentration measuring method processes FSCV data as SDBR data and provides an SDBR-applied voltammogram.

In operation 304, the neurotransmitter concentration measuring method learns the SDBR data through a deep learning network.

That is, the neurotransmitter concentration measuring method builds the deep learning model in which the SDBR data for various neurotransmitters is learned with the deep learning network.

The deep learning model processes, as SDBR data, FSCV data additionally secured by learning SDBR data that contains concentration information regardless of a time according to concentration for each substance, and supports long-time measurement of various neurotransmitters with high temporal resolution.

Hereinafter, the procedure 310 of simultaneously providing results of measuring the concentration of various neurotransmitters using the built deep learning model is described.

In operation 311, the neurotransmitter concentration measuring method collects FSCV data for measuring a neurotransmitter in the brain in real time.

In operation 312, the neurotransmitter concentration measuring method provides voltammograms before and after injecting a neurotransmitter-related drug in relation to the measured FSCV.

In operation 313, the neurotransmitter concentration measuring method processes the FSCV data as SDBR data and provides an SDBR-applied voltammogram.

In operation 314, the neurotransmitter concentration measuring method estimates changes in the concentration of various neurotransmitters through the trained deep learning model.

A graph 320 shows the changes in the concentration of various neurotransmitters estimated in operation 314.

The graph 320 may show results of simultaneously estimating the concentration of various neurotransmitters every 0.1 seconds based on the trained deep learning model.

The neurotransmitter concentration measuring method according to an example embodiment of the present invention overcomes a disadvantage of technology for measuring a change in concentration of a neurotransmitter using SDBR data, that is, a disadvantage that the concentration of only a single neurotransmitter may be measured.

Also, the neurotransmitter concentration measuring method learns the SDBR data that contains concentration information regardless of time according to the concentration for each substance and enables estimation in a not-trained electrode and environment.

The neurotransmitter concentration measuring method according to an example embodiment of the present invention can perform deep learning and then perform simultaneous estimation for all neurotransmitters that allow FSCV measurement.

Therefore, since the number of measurable neurotransmitters steadily increases through prior learning, the present invention has high development potential.

Many brain diseases are caused due to incorrect signaling change in neurotransmitters. The present invention enables deeper understanding of roles of various neurotransmitters when trying to understand a specific brain disease or brain function.

The neurotransmitter concentration measuring method according to an example embodiment of the present invention can provide a most ideal measurement environment among all existing neurotransmitter measurement technologies when trying to simultaneously observe various neurotransmitters in the brain.

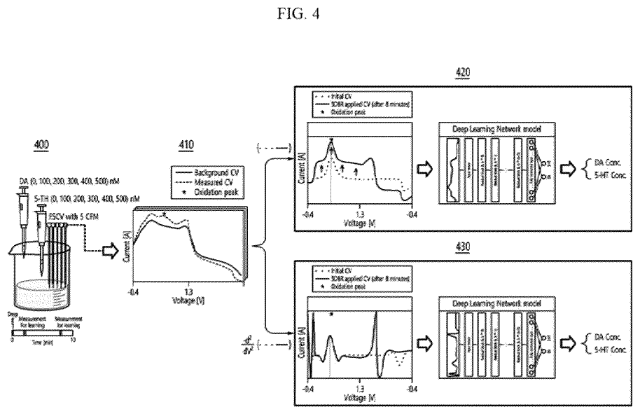

FIG. 4 is a diagram describing a concentration measurement experiment of neurotransmitters based on a deep learning model according to an example embodiment of the present invention.

FIG. 4 compares the related art and the present invention, while illustrating an invitro experiment for simultaneous estimation of dopamine and serotonin as a concentration measurement experiment of neurotransmitters based on a deep learning model according to an example embodiment of the present invention.

Referring to FIG. 4, in operations 400 and 410, a neurotransmitter concentration measuring method collects FSCV data according to a change in concentration of various neurotransmitters.

Operation 400 relates to measuring FSCV of a solution in which dopamine (DA) and serotonin (5-HT) each having different concentrations are mixed and collects FSCV data by measuring a total of 36 concentration combinations for 10 minutes with five electrodes for each concentration.

Operation 410 relates to providing the FSCV data as a voltammogram scanned every 0.1 seconds.

Operation 420 corresponds to a case in which the neurotransmitter concentration measuring method generates a deep learning model by learning data for voltage of an individual voltammogram for each scan by background subtraction in the FSCV data with a deep learning network. Here, operation 420 corresponds to the related art.

Operation 420 represents predicting the change in concentration of dopamine (DA) and serotonin (5-HT) using the deep learning network that is trained with the background-subtracted FSCV data.

Operation 430 corresponds to a case in which the neurotransmitter concentration measuring method processes the FSCV data as SDBR data in a faradaic current form in which capacitive charging current is excluded, based on a second derivative for voltage of an individual voltammogram generated for each scan by background subtraction in the FSCV data and then generates the deep learning model by learning the SDBR data with a network. Here, operation 430 corresponds to the present invention.

Operation 430 represents predicting the change in concentration of dopamine (DA) and serotonin (5-HT) using the deep learning network that is trained with the FSCV data to which SDBR is applied.

Operation 430 expresses faradaic current measured by neurotransmitter oxidation without background drift and shows that the deep learning network well estimates the change in tonic concentration when concentration information is maintained over time.

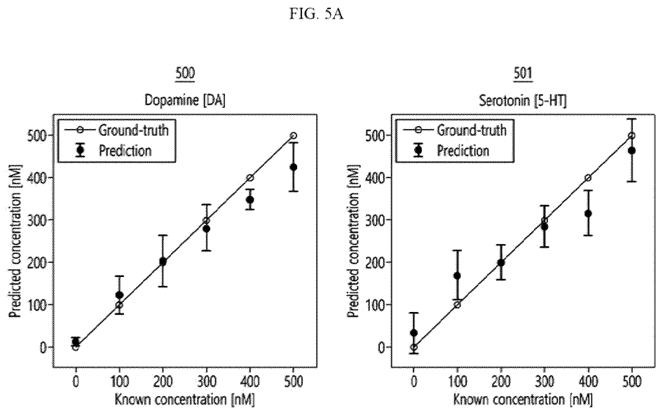

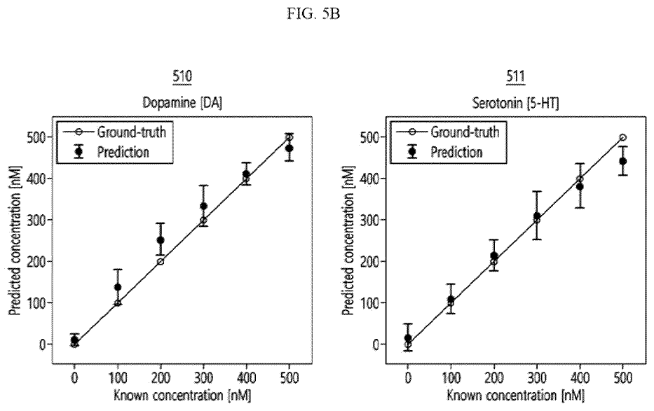

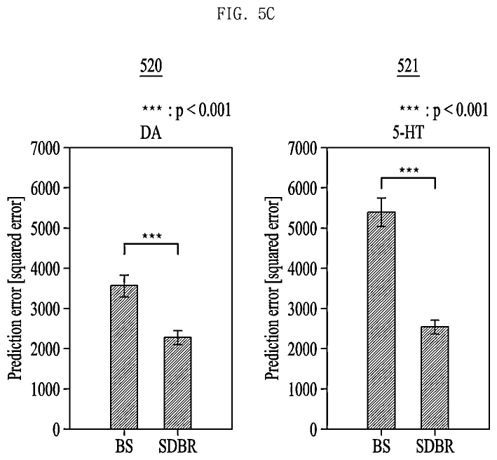

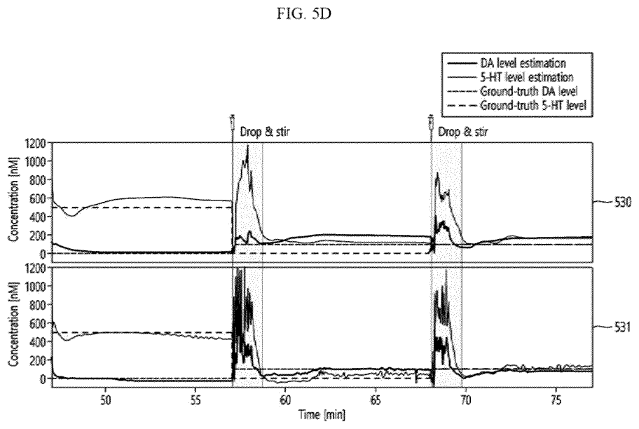

FIGS. 5A to 5D are graphs describing concentration measurement experiment results of neurotransmitters based on a deep learning model according to an example embodiment of the present invention.

FIGS. 5A and 5B illustrate the change in concentration estimated in operation 420 of FIG. 4 and the change in concentration estimated in operation 430 of FIG. 4 to describe concentration measurement experiment results of neurotransmitters based on a deep learning model according to an example embodiment of the present invention.

Referring to FIG. 5A, a graph 500 shows the change in concentration of dopamine (DA) (prediction) estimated based on a deep learning model trained through background-subtracted FSCV data, in comparison to the actual change in concentration (ground-truth).

A graph 501 shows the change in concentration of serotonin (5-HT) estimated based on the deep learning model trained through the background-subtracted FSCV data, in comparison to the actual change in concentration.

Referring to FIG. 5B, a graph 510 shows the change in concentration of dopamine (DA) estimated based on the deep learning model trained through the background-subtracted FSCV data, in comparison to the actual change in concentration.

A graph 511 shows the change in concentration of serotonin (5-HT) estimated based on the deep learning model trained through the background-subtracted FSCV data, in comparison to the actual change in concentration.

The graph 510 and the graph 511 are simultaneous measurement results and show that the actual change in concentration (ground-truth) and the estimated change in concentration (prediction) are similar.

Comparing the graph 500 and the graph 510, it can be seen that the graph 500 has low estimation accuracy compared to the graph 510.

Comparing the graph 501 and the graph 511, it can be seen that the graph 501 has low estimation accuracy compared to the graph 511.

That is, it can be verified that accuracy of simultaneously estimating dopamine and serotonin according to an example embodiment of the present invention is high.

FIG. 5C illustrates a prediction error rate for the change in concentration estimated in operation 420 of FIG. 4 and the change in concentration estimated in operation 430 of FIG. 4 for each neurotransmitter to be estimated in concentration measurement experiment results of neurotransmitters based on a deep learning model according to an example embodiment of the present invention.

Referring to FIG. 5C, a graph 520 shows a prediction error rate for dopamine (DA) and a graph 521 shows a prediction error rate for serotonin (5-HT).

The graph 520 and the graph 521 show that background subtraction (BS) according to the related art has a higher prediction error rate than SDBR corresponding to the present invention. That is, compared to the related art, the present invention may reduce a prediction error rate.

FIG. 5D compares and illustrates BS-and-SDBR-based simultaneous estimation results of dopamine and serotonin over time according to an example embodiment of the present invention.

Referring to FIG. 5D, the graph shows that SDBR has superior simultaneous estimation results compared to BS.

In the graph, BS estimation results 530 and SDBR estimation results 531 are examples of dopamine (DA) and serotonin (5-HT).

The BS estimation results 530 includes a portion in which the change in concentration does not match between estimation and ground-truth.

In the SDBR estimation results 531, the portion in which the change in concentration does not match between estimation and ground-truth appears to relatively decrease.

When comparing neurotransmitter change measurement to the related art in relation to FIGS. 5A to 5D, it may be arranged as shown in Table 1.

TABLE 1

| | [symbol] | Measurement [symbol] | | | [symbol] | [symbol] | [symbol] |
|---|---|---|---|---|---|---|---|
| Method | [symbol] | [symbol] | [symbol] | [symbol] | [symbol] | [symbol] | [symbol] |
| [symbol] | [symbol] | O | X | X | O | [symbol] | X |
| [symbol] | [symbol] | Partially | O | X | X | [symbol] | X |
| [symbol] | [symbol] | X | O | X | X | [symbol] | X |
| [symbol] | [symbol] | O | O | O | X | [symbol] | X |
| [symbol] | [symbol] | X | O | X | X | [symbol] | X |
| [symbol] | [symbol] | X | O | X | X | [symbol] | X |
| [symbol] | [symbol] | O | X | X | O | [symbol] | O |
| [symbol] | [symbol] | O | O | O | O | [symbol] | X |
| [symbol] | [symbol] | O | O | O | O | [symbol] | O |

[symbol]

indicates data missing or illegible when filed

According to Table 1, deep-FSCV according to the related art can not measure a tonic level and can not perform simultaneous measurement with a phasic level.

SDBR can measure a tonic level and can perform simultaneous measurement with a phasic level, but can not simultaneously measure multiple neurotransmitters.

Meanwhile, deep-SDBR corresponding to the present invention can measure a tonic level, can perform simultaneous measurement with a phasic level, and can simultaneously measure multiple neurotransmitters.

That is, the present invention can learn SDBR data measured in advance with various neurotransmitters using a deep learning network and can simultaneously estimate, measure, and provide the change in concentration of various neurotransmitters in real time with the trained network.

Therefore, the present invention can provide a deep-learning-based neurotransmitter concentration measuring apparatus and method for simultaneously provide long-time concentration measurement results of various neurotransmitters based on FSCV data that can measure various neurotransmitters over long time with high temporal resolution (0.1 seconds).

Also, the present invention can improve temporal resolution by extracting a faradaic current form of a neurotransmitter regardless of capacitive charging current, thereby improving a detailed analysis rate of a signal of dopamine that is a neurotransmitter.

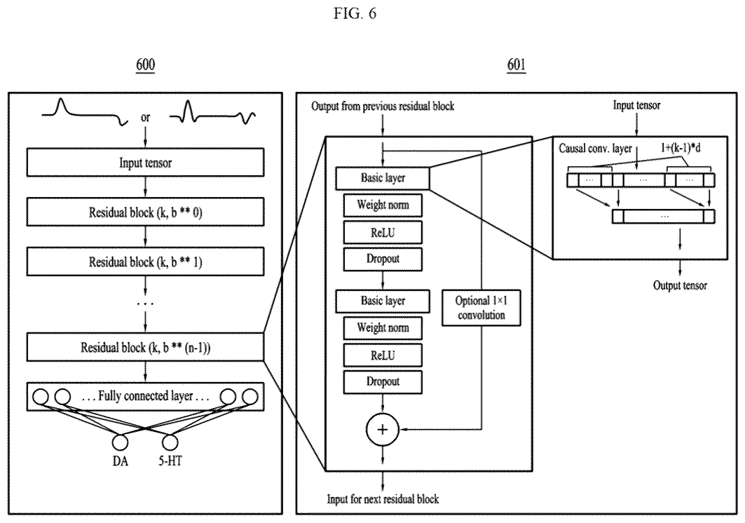

FIG. 6 is a diagram describing a deep learning model according to an example embodiment of the present invention.

FIG. 6 illustrates a deep learning model according to an example embodiment of the present invention.

Referring to FIG. 6, a block diagram 600 illustrates a temporal convolutional network (TCN) for learning time series data, which may be a deep learning model for estimating tonic dopamine and tonic serotonin levels.

Voltammograms measured for dopamine and serotonin are used as input to the TCN after BS or SDBR is applied.

To simultaneously estimate a tonic dopamine level and a tonic serotonin level, a fully connected layer is added at the end of sequence.

A block diagram 601 illustrates detailed information on a residual block of the block diagram 600.

In the block diagram 600, k denotes a size of a kernel, b denotes an expansion criterion, and cyclic voltammetry is used as input without scaling or normalization.

The fully connected layer is added at a last time stage of the sequence to estimate the concentration of dopamine and the concentration of serotonin.

The block diagram 601 is a causal convolution layer in which a basic layer is expanded. If residual input and output have different dimensions, a 1×1 convolution may be added. Here, k denotes the size of a kernel and d denotes an expansion coefficient.

FIG. 6 simply illustrates the deep learning model and the present invention is not limited to the corresponding model.

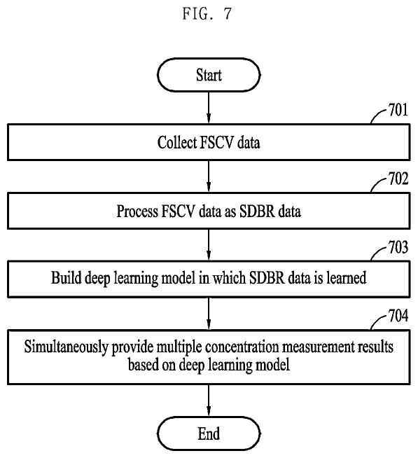

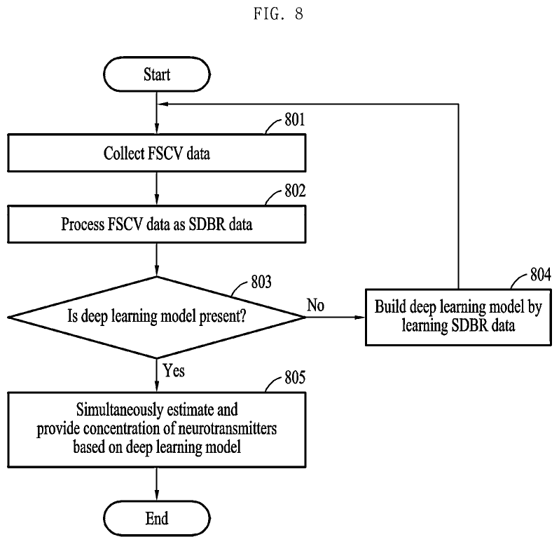

FIGS. 7 and 8 are flowcharts describing a neurotransmitter concentration measuring method according to an example embodiment of the present invention.

FIG. 7 illustrates a procedure of a neurotransmitter concentration measuring method that simultaneously measures the concentration of multiple substances by learning SDBR data with a deep learning network according to an example embodiment of the present invention.

Referring to FIG. 7, in operation 701, the neurotransmitter concentration measuring method according to an example embodiment of the present invention collects FSCV data.

That is, the neurotransmitter concentration measuring method according to an example embodiment of the present invention collects FSCV data in which capacitive charging current is reflected in faradaic current varying depending on injection concentration for each of multiple neurotransmitters.

In operation 702, the neurotransmitter concentration measuring method according to an example embodiment of the present invention processes the FSCV data as SDBR data.

That is, the neurotransmitter concentration measuring method according to an example embodiment of the present invention processes the FSCV data as the SDBR data in a faradaic current form in which the capacitive charging current is excluded based on a second derivative for voltage of an individual voltammogram generated for each scan by background subtraction in the FSCV data.

In operation 703, the neurotransmitter concentration measuring method according to an example embodiment of the present invention builds a deep learning model in which the SDBR data is learned.

That is, the neurotransmitter concentration measuring method according to an example embodiment of the present invention can build the deep learning model that simultaneously estimates concentration of multiple neurotransmitters by learning the SDBR data with a deep learning network.

In operation 704, the neurotransmitter concentration measuring method according to an example embodiment of the present invention simultaneously provides multiple concentration measurement results based on the deep learning model.

That is, the neurotransmitter concentration measuring method according to an example embodiment of the present invention can simultaneously provide concentration measurement results of a neurotransmitter varying in response to real-time injection for each of multiple neurotransmitters based on the deep learning model.

FIG. 8 illustrates a procedure of a neurotransmitter concentration measuring method that learns SDBR data with a deep learning network and simultaneously measures the concentration of multiple substances by dividing the procedure into learning and measurement according to an example embodiment of the present invention.

Referring to FIG. 8, in operation 801, the neurotransmitter concentration measuring method according to an example embodiment of the present invention collects FSCV data.

In operation 802, the neurotransmitter concentration measuring method according to an example embodiment of the present invention processes the FSCV data as SDBR data.

That is, the neurotransmitter concentration measuring method according to an example embodiment of the present invention generates SDBR data corresponding to each piece of FSCV data by performing SDBR processing on the FSCV data for multiple neurotransmitters.

In operation 803, the neurotransmitter concentration measuring method according to an example embodiment of the present invention determines whether a deep learning model for corresponding SDBR data is present.

That is, the neurotransmitter concentration measuring method can update the deep learning model by considering a learning status of new SDBR data and previously learned SDBR data for expansion of the deep learning model.

The neurotransmitter concentration measuring method according to an example embodiment of the present invention performs operation 805 when the deep learning model for the SDBR data is present and, otherwise, performs operation 804.

In operation 804, the neurotransmitter concentration measuring method according to an example embodiment of the present invention builds the deep learning model by learning the SDBR data.

In operation 805, the neurotransmitter concentration measuring method according to an example embodiment of the present invention estimates the concentration of neurotransmitters based on the built deep learning model and provides the change in the estimated concentration.

Therefore, the present invention can continuously increase the number of simultaneously measurable neurotransmitters through pre-training by enabling simultaneous estimation after deep learning model training for all neurotransmitters that can be measured in FSCV data.

The apparatuses described herein may be implemented using hardware components, software components, or a combination of the hardware components and the software components. For example, the apparatuses and the components described herein may be implemented using one or more general-purpose or special purpose computers, such as, for example, a processor, a controller, an arithmetic logic unit (ALU), a digital signal processor, a microcomputer, a field programmable array (FPA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that the processing device may include multiple processing elements and/or multiple types of processing elements. For example, the processing device may include multiple processors or a processor and a controller. In addition, different processing configurations are possible, such as parallel processors.

The methods according to the above-described example embodiments may be configured in a form of program instructions performed through various computer devices and recorded in computer-readable media. The media may include, alone or in combination with program instructions, data files, data structures, and the like. The program instructions recorded in the media may be specially designed and configured for the example embodiments, or may be known and available to those skilled in the computer software art. Examples of the media include magnetic media such as hard disks, floppy disks, and magnetic tapes; optical media such as CD-ROM and DVDs; magneto-optical media such as floptical disks; and hardware devices that are configured to store program instructions, such as read-only memory (ROM), random access memory (RAM), flash memory, and the like. Examples of the program instructions include a machine language code as produced by a compiler and an advanced language code executable by a computer using an interpreter. The hardware device may be configured to operate as at least one software module, or vice versa.

The software may include a computer program, a piece of code, an instruction, or some combinations thereof, for independently or collectively instructing or configuring the processing device to operate as desired. Software and/or data may be permanently or temporarily embodied in any type of machine, component, physical equipment, virtual equipment, a computer storage medium or device, or a signal wave to be transmitted to be interpreted by the processing device or to provide an instruction or data to the processing device. The software also may be distributed over network coupled computer systems so that the software is stored and executed in a distributed fashion. The software and data may be stored by one or more computer readable storage media.

Although the example embodiments are described with reference to the accompanying drawings, it will be apparent to one of ordinary skill in the art that various alterations and modifications in form and details may be made in these example embodiments without departing from the spirit and scope of the claims and their equivalents. For example, suitable results may be achieved if the described techniques are performed in different order, and/or if components in a described system, architecture, device, or circuit are combined in a different manner, and/or replaced or supplemented by other components or their equivalents.

Therefore, other implementations, other example embodiments, and equivalents of the claims are to be construed as being included in the claims.

1. A neurotransmitter concentration measuring apparatus comprising:
a data collecting unit configured to collect fast-scan cyclic voltammetry (FSCV) data in which capacitive charging current is included in faradaic current varying depending on injection concentration for each of multiple neurotransmitters;
a data processing unit configured to process the FSCV data as second-derivative-based background removal (SDBR) data in a faradaic current form in which the capacitive charging current is excluded, based on a second derivative for voltage of an individual voltammogram generated for each scan by background subtraction in the FSCV data;
a deep learning processing unit configured to build a deep learning model that simultaneously estimates the concentration of the multiple neurotransmitters by learning the SDBR data with a deep learning network; and
a measurement result providing unit configured to simultaneously provide concentration measurement results of a neurotransmitter varying depending on real-time injection for each of the multiple neurotransmitters based on the deep learning model.

2. The neurotransmitter concentration measuring apparatus of claim 1, wherein the deep learning processing unit is configured to build the deep learning model that simultaneously estimates the concentration of the multiple neurotransmitters when SDBR data based on FSCV data additionally measured for one neurotransmitter among the multiple neurotransmitters is applied to the deep learning model built by learning SDBR data that is measured and processed in advance for the multiple neurotransmitters with the deep learning network.

3. The neurotransmitter concentration measuring apparatus of claim 1, wherein the multiple neurotransmitters include at least two of dopamine, epinephrine, norepinephrine, and serotonin.

4. The neurotransmitter concentration measuring apparatus of claim 1, wherein the data processing unit is configured to process the FSCV data as the SDBR data by extracting, from the FSCV data, the individual voltammogram in which the faradaic current and the capacitive charging current are reflected together for each scan by the background subtraction in relation to a peak according to neurotransmitter injection, by multiplying voltage of the extracted individual voltammogram by a negative value after the second derivative, and by quantifying a curvature of a neurotransmitter peak.

5. The neurotransmitter concentration measuring apparatus of claim 4, wherein the extracted individual voltammogram includes phasic measurement results in relation to the neurotransmitter concentration measurement, and
the SDBR data includes the phasic measurement results and tonic measurement results in relation to the neurotransmitter concentration measurement.

6. The neurotransmitter concentration measuring apparatus of claim 1, wherein the data processing unit is configured to extract the individual voltammogram such that a voltammogram around a neurotransmitter oxidation peak after the background subtraction has a symmetrical Gaussian shape.

7. The neurotransmitter concentration measuring apparatus of claim 6, wherein the data processing unit is configured to process the SDBR data such that amplitude current of a neurotransmitter oxidation peak of a voltammogram corresponding to the individual voltammogram has a linear correlation with the concentration of a neurotransmitter and background charging current generated around the neurotransmitter oxidation peak is irrelevant to voltage.

8. The neurotransmitter concentration measuring apparatus of claim 1, wherein the measurement result providing unit is configured to determine a neurotransmitter oxidation peak voltage based on the faradaic current form in which the capacitive charging current based on the SDBR data is excluded and to provide the concentration of a neurotransmitter compared to the determined neurotransmitter oxidation peak voltage as the concentration measurement results of the neurotransmitter.

9. The neurotransmitter concentration measuring apparatus of claim 1, wherein the data collecting unit is configured to collect the FSCV data in which the faradaic current increasing at an injection point in time of the neurotransmitter and the capacitive charging current gradually increasing over time are combined.

10. A neurotransmitter concentration measuring method comprising:
collecting, by a data collecting unit, fast-scan cyclic voltammetry (FSCV) data in which capacitive charging current is reflected in faradaic current varying depending on injection concentration for each of multiple neurotransmitters;
processing, by a data processing unit, the FSCV data as second-derivative-based background removal (SDBR) data in a faradaic current form in which the capacitive charging current is excluded, based on a second derivative for voltage of an individual voltammogram generated for each scan by background subtraction in the FSCV data;
building, by a deep learning processing unit, a deep learning model that simultaneously estimates the concentration of the multiple neurotransmitters by learning the SDBR data with a deep learning network; and
simultaneously providing, by a measurement result providing unit, concentration measurement results of a neurotransmitter varying depending on real-time injection for each of the multiple neurotransmitters based on the deep learning model.

11. The neurotransmitter concentration measuring method of claim 10, wherein the building of the deep learning model that simultaneously estimates the concentration of the multiple neurotransmitters by learning the SDBR data with the deep learning network comprises building the deep learning model that simultaneously estimates the concentration of the multiple neurotransmitters when SDBR data based on FSCV data additionally measured for one neurotransmitter among the multiple neurotransmitters is applied to the deep learning model built by learning SDBR data that is measured and processed in advance for the multiple neurotransmitters with the deep learning network.

12. The neurotransmitter concentration measuring method of claim 10, wherein the multiple neurotransmitters include at least two of dopamine, epinephrine, norepinephrine, and serotonin.

13. The neurotransmitter concentration measuring method of claim 10, wherein the processing of the FSCV data as the SDBR data in the faradaic current form in which the capacitive charging current is excluded, based on the second derivative for voltage of the individual voltammogram generated for each scan by background subtraction in the FSCV data comprises processing the FSCV data as the SDBR data by extracting, from the FSCV data, the individual voltammogram in which the faradaic current and the capacitive charging current are reflected together for each scan by the background subtraction in relation to a peak according to neurotransmitter injection, by multiplying voltage of the extracted individual voltammogram by a negative value after the second derivative, and by quantifying a curvature of a neurotransmitter peak.

14. The neurotransmitter concentration measuring method of claim 13, wherein the extracted individual voltammogram includes phasic measurement results in relation to the neurotransmitter concentration measurement, and
the SDBR data includes the phasic measurement results and tonic measurement results in relation to the neurotransmitter concentration measurement.
